(19) United States
(12) Patent Application Publication
(10) Pub. No.: US 2018/0044725 A1
(43) Pub. Date: Feb. 15, 2018

(54) POLYNUCLEOTIDE BINDING PROTEIN SEQUENCING

(71) Applicant: Stratos Genomics, Inc., Seattle, WA (US)

(72) Inventors: Mark Stamatios Kokoris, Bothell, WA (US); Robert N. McRuer, Mercer Island, WA (US)

(21) Appl. No.: 15/555,438

(22) PCT Filed: Mar. 3, 2016
(86) PCT No.: PCT/US16/20752

§ 371 Date: Sep. 1, 2017

Related U.S. Application Data
(60) Provisional application No. 62/127,464, filed on Mar. 3, 2015.

(51) Int. Cl. C12Q 1/68; G01N 27/447; G01N 33/487

(52) U.S. Cl. CPC C12Q 1/6869; G01N 33/48721; G01N 27/44791; G01N 27/44726

(57) ABSTRACT

The present disclosure relates to systems, methods and compositions for single molecule electronic sequencing of template nucleic acids using nanosensors. The nanosensors of the invention improve the measurement of polynucleotides by assimilating authentic polynucleotide binding proteins (“PBPs”) in place of conventional pore-forming proteins that do not normally interact with polynucleotides. The PBPs of the present invention form the constriction sites of electroconductive pores of the nano sensors, while maintaining their natural polynucleotide binding and processing activities.

### BACKGROUND

### Technical Field

The present invention embodiments relate generally to the field of biosensors. More specifically, the compositions and methods describe herein relate to a polynucleotide binding protein (PBP) that can be assimilated with a membrane to form an electroconductive aperture for use in DNA sequencing and other applications.

### Description of the Related Art

Nucleic acid sequences encode the necessary information for living organism to function and reproduce and are in essence a blueprint for life. The ability to determine such sequences is therefore a tool useful in basic research into how and where organisms live, as well as in applied sciences, such as drug development. In medicine, sequencing tools can be used for diagnosis and to develop treatments for a variety of pathologies, including cancer, heart disease, autoimmune disorders, multiple sclerosis, and obesity. In industry, sequencing can be used to design improved enzymatic processes or synthetic organisms. In biology, such tools can be used to study the health of ecosystems, for example, and thus have a broad range of utility.

An individual's unique DNA sequence provides valuable information concerning their susceptibility to certain diseases. The sequence has the potential to provide patients with the opportunity to screen for early detection and to receive preventative treatment. Furthermore, given a patient's individual blueprint, clinicians have the opportunity to administer personalized therapy to maximize drug efficacy and to minimize the risk of an adverse drug response. Similarly, determining the blueprint of pathogenic organisms has the potential to lead to new treatments for infectious diseases and more robust pathogen surveillance. Low cost, whole genome DNA sequencing will thus provide the foundation for modern medicine. To achieve this goal, sequencing technologies must continue to advance with respect to throughput, accuracy, and read length.

During recent years, several next generation DNA sequencing technologies have become commercially available that have dramatically reduced the cost of whole genome sequencing. These include sequencing by synthesis (“SBS”) platforms (e.g., those developed by Illumina, Inc., 454 Life Sciences, Ion Torrent, and Pacific Biosciences) and analogous ligation-based platforms (e.g., those developed by Complete Genomics and Life Technologies Corp.). A number of other technologies have been in development that utilize a wide variety of sample processing and detection methods. For example, GnuBIO, Inc. is developing a system that uses picoliter reaction vessels to control millions of discreet probe sequencing reactions, whereas Halcyon Molecular has described development of technology for direct DNA measurement using a transmission electron microscope.

Nanopore-based nucleic acid sequencing is a compelling approach that has been widely studied. In pioneering studies, Kasianowicz and colleagues characterized single-stranded polynucleotides as they were electrically translocated through an alpha hemolysin nanopore embedded in a lipid bilayer (see, e.g., Kasianowicz, J. (1996). Characterization of Individual Polynucleotide Molecules using a Membrane Channel. Proc. Natl. Acad. Sci., 93, 13770-3). It was demonstrated that during polynucleotide translocation partial blockage of the nanopore aperture could be measured as a decrease in ionic current. However, polynucleotide sequencing in nanopore is burdened by the need to resolve tightly-spaced nucleotides (e.g., 0.34 nm) with small signal differences immersed in significant background noise. The measurement challenge of single base resolution in a nanopore is made more demanding due to the rapid translocation rates observed for polynucleotides, which are typically on the order of one base per microsecond. Translocation rate can be reduced by adjusting run parameters, such as voltage, salt concentration, pH, temperature, and viscosity to name a few. However, such adjustments have been unable to reduce translocation rate to a level that allows for single base resolution.

Stratos Genomics is developing a method called Sequencing by Expansion (“SBX”) that uses a biochemical process to transcribe the sequence of a DNA molecule onto a measurable polymer called an “Xpandomer” (see, e.g., U.S. Pat. No. 7,939,259 to Kokoris et al.). The transcribed sequence is encoded along the Xpandomer backbone in high signal-to-noise reporters that are separated by approximately 10 nm and are designed for high signal-to-noise, well-differentiated responses. These differences provide significant performance enhancements in sequence read efficiency and accuracy of Xpandomers relative to native DNA. Xpandomers can enable several next generation DNA sequencing detection technologies but are well suited to nanopore sequencing.

Gundlach and colleagues have demonstrated a method of sequencing DNA that uses a low noise nanopore derived from Mycobacterium smegmatis (“MspA”) in conjunction with a process called duplex interrupted sequencing (see, e.g., Derrington, I. et al. (2010). Nanopore DNA Sequencing with MspA. Proc. Natl. Acad. Sci., 107(37), 16060-16065). In short, a double strand duplex is used to temporarily hold the single-stranded portion of the nucleic acid in the MspA constriction. This process enables better statistical sampling of the bases held in the limiting aperture. Under such conditions, single base identification was demonstrated, however, this approach requires a DNA conversion method, such as those disclosed by Kokoris et al.

Akeson and colleagues (see, e.g., PCT Publication No. WO/20150344945) have disclosed methods for characterizing polynucleotides in a nanopore that utilize an adjacently positioned molecular motor to control the translocation rate of the polynucleotide through or adjacent to the nanopore aperture. At this controlled translocation rate (with an implied measurement rate of 350-2000 Hz), the signal corresponding to the movement of the target polynucleotide with respect to the nanopore aperture can be more closely correlated to the identity of the bases within and proximal to the aperture constriction. Even with molecular motor control of polynucleotide translocation rate through a nanopore, single base measurement resolution is still limited to the dimension and composition of the aperture constriction. As such, in separate work, Bayley and colleagues (using an alpha-hemolysin nanopore system) and Gundlach and colleagues (using an MspA nanopore system) have disclosed methods for engineering nanopores with enhanced noise and base resolution characteristics. However, a demonstration of processive individual nucleotide sequencing has yet to be published that uses either (or both) a molecular motor for translocation control and an engineered nanopore. Current state of the art suggests that signal deconvolution of at least triplet base sets is required in order to assign single base identity.

Indeed, nanopores have proved to be powerful amplifiers, much like their highly exploited predecessors, the Coulter Counters. However, the current generation of organic nanopores (e.g., alpha-hemolysin and MspA) that have been tasked with base recognition of DNA are transmembrane proteins that do not naturally interact with DNA. As such, they do not have natural functions for controlling DNA translocation. As has been discussed, this is a recognized shorting that some have attempted to overcome by adding functionality with protein motors adjacent to the nanopores. In another example, Akeson and colleagues added phi29 polymerase adjacent to the alpha hemolysin nanopore so that single-stranded DNA could be fed into the pore at a controlled rate (see, e.g., Cherf, G. M. et al. (2012). Automated Forward and Reverse Ratcheting of DNA in a Nanopore at 5 Å Precision. Nat. Biotech. 30, 344-348). This approach complicates the assay and imposes a separation of the measurement region in the alpha hemolysin nanopore from the position control in the polymerase that can introduce additional noise and sequence-dependent variation to the measurement.

Clearly, there is a need for improved compositions and methods that would provide a versatile membrane conductive channel platform for efficiently and sensitively determining the sequence of nucleic acids and whole genomes. The presently disclosed invention embodiments address such needs, and offer other related advantages.

### BRIEF SUMMARY

The invention provides systems, methods, and compositions for sequencing nucleic acids using nanosensors. The nanosensors of the present invention are active nanopores that improve the measurement of polynucleotides by replacing conventional pore-forming proteins that do not normally interact with polynucleotides with authentic polynucleotide binding proteins (“PBPs”). The PBPs of the present invention form ion current constriction sites in the electroconductive pores of the nanosensors that actively change during the PBP's natural polynucleotide binding and/or processing activities.

In one aspect, the invention provides a system for determining the nucleotide sequence of a polynucleotide in a sample including a cis chamber and a trans chamber, in which the cis and trans chamber are separated by a membrane, and in which the cis and trans chambers include an electrically conductive media; a polynucleotide binding protein assimilated with the membrane to form an electroconductive pore therein, in which the polynucleotide binding protein provides a constriction site in the pore and in which the constriction site undergoes conformational changes in response to processing of a target polynucleotide by the polynucleotide binding protein; drive electrodes in contact with the reaction mixture on either side of the membrane for providing a voltage drop across the pore; one or more measurement electrodes connected to electronic measurement equipment for measuring ion current through the pore; and a computer for identifying the types of nucleotides sequentially processed by the polynucleotide binding protein. In some embodiments, the reaction mixture includes reagents necessary for polynucleotide processing. In certain embodiments, the polynucleotide binding protein is a helicase, a DNA polymerase, a RNA polymerase, an exonuclease, and endonuclease, or a transcription factor. In one particular embodiment, the polynucleotide binding protein is a helicase or a DnaB-like. In another embodiment, the polynucleotide binding protein is an exonuclease. In yet another embodiment, the polynucleotide binding protein is a DNA polymerase. In other embodiments, the membrane is composed of amphiphilic molecules. In yet other embodiments, the amphiphilic molecules form a lipid bilayer. In other embodiments, the membrane is a solid-state membrane. In some embodiments, the polynucleotide binding protein is assimilated with a pore preformed in the solid-state membrane. In other embodiments, the polynucleotide binding protein is assimilated with a support pore embedded in the lipid bilayer. In another embodiment, the support pore is a natural pore forming protein. In another embodiment, the polynucleotide binding protein is assimilated with the lipid bilayer by embedding the protein in the bilayer. In certain embodiments, the polynucleotide binding protein in genetically modified to introduce hydrophobic groups on at least one outer surface of the protein.

In another aspect, the invention provides a method for determining sequence information about a nucleic acid molecule including the steps of providing a membrane with at least one polynucleotide binding protein assimilated therein to form an electroconductive pore, in which the polynucleotide binding protein provides a constriction site in the pore, and in which the constriction site undergoes conformational changes in response to binding and processing of a target polynucleotide by the polynucleotide binding protein; contacting the polynucleotide binding protein with a reaction mixture including the reagents required for electrical conductance and polynucleotide processing by the polynucleotide binding protein; providing a voltage drop across the constriction site that induces ion current through the pore that is modulated by polynucleotide processing of the membrane spanning polynucleotide; measuring the resulting base specific ion current over, thus determining the sequence information of the polynucleotide. In some embodiments, the resulting base specific ion current may include the magnitude of the ion current through the constriction site or the shape of the measured ion current over time. In other embodiments, the polynucleotide binding protein may be a helicase, a DNA polymerase, a RNA polymerase, an exonuclease, an endonuclease, or a transcription factor. In certain embodiments, the polynucleotide binding protein is a helicase or a DnaB-like helicase. In some embodiments, the polynucleotide is a double-stranded nucleic acid. In other embodiments, the polynucleotide binding protein is an exonuclease. In further embodiments, the polynucleotide is a double-stranded nucleic acid. In yet other embodiments, the polynucleotide binding protein is a DNA polymerase. In another embodiment, the polynucleotide is an oligonucleotide primer bound to a single stranded nucleic acid template. In other embodiments, the membrane is composed of amphiphilic molecules. In yet other embodiments, the amphiphilic molecules form a lipid bilayer. In other embodiments, the membrane is a solid-state membrane. In some embodiments, the polynucleotide binding protein is assimilated with a pore preformed in the solid-state membrane. In other embodiments, the polynucleotide binding protein is assimilated with a support pore embedded in the lipid bilayer. In another embodiment, the support pore is a natural pore forming protein. In another embodiment, the polynucleotide binding protein is assimilated with the lipid bilayer by embedding the protein in the bilayer. In certain embodiments, the polynucleotide binding protein in genetically modified to introduce hydrophobic groups on at least one outer surface of the protein. In another aspect, the invention provides methods for determining the nucleotide sequence of a polynucleotide in a sample, including the steps of providing a sold-state membrane having at least one polynucleotide binding protein assimilated therein to form an electroconductive pore, in which the polynucleotide binding protein provides a constriction site in the pore, and in which the constriction site undergoes conformational changes in response to processing of a target polynucleotide by the polynucleotide binding protein; contacting the polynucleotide binding protein with a reaction mixture including the reagents required for electrical conductance and polynucleotide processing by the polynucleotide binding protein; providing a high frequency drive potential across the membrane; measuring conductivity through the constriction site over time to detect the nucleotide-dependent binding and modification; and identifying the types of nucleotides bound and processed by the polynucleotide binding protein, thus determining sequence information about the polynucleotide.

In another aspect, the invention provides methods for determining the nucleotide sequence of a polynucleotide in a sample, including the steps of providing a membrane having at least one polynucleotide binding protein assimilated therein to form an electroconductive pore, in which the polynucleotide binding protein provides a constriction site in the pore, and in which the protein is complexed with a target polynucleotide; contacting the polynucleotide binding protein with a reaction mixture including the reagents required polynucleotide processing by the polynucleotide binding protein; providing an optically detectable agent to the reaction mixture on a first side of the membrane, in which the agent is capable of flowing through the pore to the reaction mixture on a second side of the membrane; measuring the concentration of the agent in the reaction mixture on the second side of the membrane over time to detect the nucleotide-dependent binding and processing using optical means; and identifying the types of nucleotides processed by the polynucleotide binding protein using concentration modulation characteristics, thus determining sequence information about the polynucleotide. In some embodiments, the optical means measure the agent directly. In other embodiments, the agent is fluorescein. In yet other embodiments, the optical means measure the agent indirectly. In further embodiments, the agent is calcium. In other embodiments, the reaction mixture on the second side of the membrane includes a fluorescent calcium indicator probes. In yet other embodiments, the fluorescent calcium indicator probe is Fluo-3, Fluo-4, or Fluo-5. In other embodiments, the membrane is composed of amphiphilic molecules. In yet other embodiments, the amphiphilic molecules form a lipid bilayer. In other embodiments, the membrane is a solid-state membrane. In some embodiments, the polynucleotide binding protein is assimilated with a pore preformed in the solid-state membrane. In other embodiments, the polynucleotide binding protein is assimilated with a support pore embedded in the lipid bilayer. In another embodiment, the support pore is a natural pore forming protein. In another embodiment, the polynucleotide binding protein is assimilated with the lipid bilayer by embedding the protein in the bilayer. In certain embodiments, the polynucleotide binding protein in genetically modified to introduce hydrophobic groups on at least one outer surface of the protein.

### BRIEF DESCRIPTION OF THE SEVERAL VIEWS OF THE DRAWINGS

In the figures, the sizes and relative positions of elements are not necessarily drawn to scale and some of these elements are arbitrarily enlarged and positioned to improve figure legibility. Further, the particular shapes of the elements as drawn are not intended to convey any information regarding the actual shape of the particular elements, and have been solely selected for ease of recognition in the figures.

FIG. 1 shows a generic cartoon of one embodiment of a PBP nanosensor of the invention.

FIG. 2 shows a cartoon of another embodiment of a PBP nanosensor of the invention, in which a helicase enzyme is associated with a solid-state membrane.

FIG. 3 shows a cartoon of another embodiment of a PBP nanosensor of the invention, in which an exonuclease enzyme is embedded in a membrane.

FIG. 4 shows a cartoon of another embodiment of a PBP nanosensor of the invention, in which a PBP is associated with a solid-state membrane and measurements are taken of electrical fringe fields.

FIGS. 5A-C show cartoons of alternative means of assimilating PBPs with membranes to form the nanosensors of the present invention.

FIG. 6 shows a cartoon of a solid-state sensor chip of the present invention.

FIG. 7 depicts an electron micrographic image of an electroconductive pore drilled into a solid-state silicon chip

### DETAILED DESCRIPTION

### Definitions

The term “electroconductive pore,” as used herein, generally refers a structure that conducts current from one reservoir to another. An electroconductive pore may form or otherwise provide a pore, channel, aperture, or passage in a membrane that permits hydrated ions to flow from one side of a membrane to the other side of the membrane. An electroconductive pore can be defined by a molecule in a membrane, or other suitable substrate. The structure forming the electroconductive pore may be referred to as a transmembrane pore and may be defined by a multiple of smaller pores within a defined boundary acting collectively like a single pore. Transmembrane pores may also be referred to as “nanopores”.

The transmembrane, or electroconductive, pores of the present invention are formed by proteins and may be a single polypeptide or a collection of polypeptides made up of several repeating subunits. Protein transmembrane pores may not function naturally as transmembrane pores. Protein transmembrane pores that do not function naturally as include the polynucleotide binding proteins described herein. Transmembrane pores typically cross the entire membrane so that hydrated ions may flow through an electroconductive aperture from one side of the membrane to the other side of the membrane. However, the aperture (i.e. channel) formed by the transmembrane pore does not have to cross the membrane, e.g., it may be closed at one end and transiently opened due to conformational changes in the protein under suitable conditions. The electroconductive pore may be formed from a support pore and a second pore-forming protein, e.g. a polynucleotide binding protein as described herein. When an electroconductive pore includes a support pore and a second pore-forming protein, the second pore-forming protein typically forms, or provides, the constriction site of the transmembrane pore.

The term “constriction site”, or “constriction zone”, as used herein refers to a narrow portion of the aperture, or channel, formed by the polynucleotide binding protein that modulates ion current passing through it due to the protein's processing of the target polynucleotide. The constriction site is thus a narrow three dimensional region in the interior of the pore that undergoes conformational change during polynucleotide processing. The conformational changes in the constriction site modulates passage of electrolytes, and thus the current output signal, can vary. The constriction site may also be formed from a collection of small conduits, the current through each of which is modulated by conformational changes in the PBP during polynucleotide processing. For example, the collection of conduits may include an aperture in the PBP itself and one or more apertures formed between the PBP and one or more luminal surfaces of a support pore. The output signal produced by the transmembrane pore systems of the present invention is any measurable signal that provides a multitude of distinct and reproducible signals depending on the physical characteristics (e.g. conformation) of the pore polypeptide and substrate molecules bound in the constriction site. A transmembrane pore may be disposed adjacent or in proximity to a sensing circuit, such as, for example, a complementary metal-oxide semiconductor (CMOS) or field effect transistor (FET) circuit.

The term “polynucleotide binding protein,” as used herein, generally refers to any protein that is capable of binding to a polynucleotide and controlling its movement with respect to a pore, such as through the pore. It is straightforward in the art to determine whether or not a protein binds to a polynucleotide. The polynucleotide binding protein typically interacts with and modifies (i.e. processes) at least one property of a polynucleotide. Processing of the polynucleotide may also include orienting it or moving it to a specific position.

Polynucleotide binding proteins of the present invention are preferably derived from a polynucleotide handling, or processing, enzyme. A polynucleotide processing enzyme is a polypeptide that is capable of interacting with and modifying, or processing, at least one property of a polynucleotide. The protein may process the polynucleotide by unwinding the strands of a double helix to form regions of single-stranded DNA. In other embodiments, the protein may process the polynucleotide by cleaving it to form individual nucleotides. The polynucleotide processing enzyme undergoes conformational changes as it acts upon its substrate polynucleotide during nucleic acid processing. The term “conformational change,” as used herein, when used in reference to polynucleotide binding proteins, means at least one change in the structure of the protein, a change in the shape of the protein or a change in the arrangement of parts of the protein. The protein can be, for example, a helicase, exonuclease, transcription factor or other nucleic acid handling protein, such as those set forth herein below. The parts of the protein can be, for example, atoms that change relative location due to rotation about one or more chemical bonds occurring in the molecular structure between the atoms. The parts can also be regions of secondary, tertiary or quaternary structure. The parts of the protein can further be domains of a macromolecule, such as those commonly known in the relevant art.

Preferred polynucleotide binding proteins are helicases, DNA and RNA polymerases, endo- and exonucleases, and transcription factors. Suitable helicases include, but are not limited to, the bacteriophage proteins, T7 gp4, T4 gp41, and Dda, the E. coli proteins, UvrD, DnaB, RuvB, rho factor, RecD, RecQ, TRCF, and Rep, the Staphylococcus aureus protein PcrA, viral proteins, NS3, LTag, E1, and Rep, the F-plasmid protein, Tral, the yeast protein, eIF4A, and the human protein, WRN. Suitable polymerases include, but are not limited to, DNA-dependent DNA polymerases, DNA-dependent RNA polymerases, RNA-dependent DNA polymerases, RNA-dependent RNA polymerases, T7 DNA polymerase, T3 DNA polymerase, T4 DNA polymerase, T7 RNA polymerase, T3 RNA polymerase, SP6 RNA polymerase, DNA polymerase I, Klenow fragment, Thermophilus aquaticus DNA polymerase, Tth DNA polymerase, VentR® DNA polymerase (New England Biolabs), Deep Vent® DNA polymerase (New England Biolabs), Bst DNA polymerase large fragment, Stoeffel fragment, 9oN DNA polymerase, Pfu DNA polymerase, Tfl DNA polymerase, Tth DNA polymerase, RepliPHI Phi29 polymerase, Tli DNA polymerase, eukaryotic DNA polymerase beta, telomerase, Therminator™ polymerase (New England Biolabs), KOD HiFi™ DNA polymerase (Novagen), KOD1 DNA polymerase, Q-beta replicase, terminal transferase, AMV reverse transcriptase, M-MLV reverse transcriptase, Phi6 reverse transcriptase, HIV-1 reverse transcriptase, novel polymerases discovered by bioprospecting, and polymerases cited in U.S. Patent Application No. US2007/0048748, U.S. Pat. Nos. 6,329,178, 6,602,695, and 6,395,524 (incorporated herein by reference in their entireties). These polymerases include wild-type, mutant isoforms, and genetically engineered variants. Suitable exonucleases include, but are not limited to, exonuclease Lambda, T7 exonuclease, exonuclease V (RecBCD), Exo III, ReCj1 exonuclease, exonuclease I, and Exo T. Suitable transcription factors include, but are not limited to, XPB and ERCC2A “membrane,” as used herein, is a thin film or other structure or interface that separates two compartments or reservoirs and prevents the free diffusion of ions and other molecules between these. Suitable membranes are amphiphilic layers formed of amphiphilic molecules, i.e. molecules possessing both hydrophilic and lipophilic properties. Such amphiphilic molecules may be either naturally occurring, such as phospholipids, or synthetic. Examples of synthetic amphiphilic molecules include such molecules as poly (n-butyl methacrylate-phosphorylcholine), poly (ester amide)-phosphorylcholine, polylactide-phosphorylcholine, polyethylene glycol-poly(caprolactone)-di- or tri-blocks, polyethylene glycol-polylactide di- or tri-blocks and polyethylene glycol-poly(lactide-glycolide) di-or tri-blocks. Preferably, the amphiphilic layer is a lipid bilayer. Lipids bilayers are models of cell membranes and have been widely used for experimental purposes. A membrane can also be a solid-state membrane, i.e. a layer prepared from solid-state materials in which one or more aperture is formed. The membrane may be a layer, such as a coating or film on a supporting substrate, or it may be a free-standing element.

“Nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N-6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, La.), all herein incorporated by reference in their entireties.

“Nucleobase residue” includes nucleotides, nucleosides, fragments thereof, and related molecules having the property of binding to a complementary nucleotide. Deoxynucleotides and ribonucleotides, and their various analogs, are contemplated within the scope of this definition. Nucleobase residues may be members of oligomers and probes. “Nucleobase”, “nucleobase residue” and “nucleotide” may be used interchangeably herein and are generally synonymous unless context dictates otherwise.

“Polynucleotides”, also called nucleic acids, are covalently linked series of nucleotides in which the 3′ position of the pentose of one nucleotide is joined by a phosphodiester group to the 5′ position of the next. DNA (deoxyribonucleic acid) and RNA (ribonucleic acid) are biologically occurring polynucleotides in which the nucleotide residues are linked in a specific sequence by phosphodiester linkages. As used herein, the terms “polynucleotide” or “oligonucleotide” encompass any polymer compound having a linear backbone of nucleotides. Oligonucleotides are generally shorter chained polynucleotides. Nucleic acid are generally referred to as “target nucleic acid” if targeted for sequencing.

The polynucleotides may be any length. For example, the polynucleotide can be at least 10, at least 50, at least 100, at least 150, at least 200, at least 250, at least 300, at least 400, or at least 500 nucleotides in length. The polynucleotide can be 1000 or more nucleotides in length, 5000 or more nucleotides in length, or 100,000 or more nucleotides in length. The polynucleotides may be single stranded, double stranded, or have regions that are single stranded and regions that are double stranded.

“Nucleic acid” is a polynucleotide or an oligonucleotide. A nucleic acid molecule can be deoxyribonucleic acid (DNA), ribonucleic acid (RNA), or a combination of both. Nucleic acids are generally referred to as “target nucleic acids” or “target sequence” if targeted for sequencing. Nucleic acids can be mixtures or pools of molecules targeted for sequencing.

The articles “a”, “an” and “the” are non-limiting. For example, “the method” includes the broadest definition of the meaning of the phrase, which can be more than one method and reference to “an enzyme” may include two or more enzymes.

All publications, patents, and patent applications cited herein, whether supra or infra, are hereby incorporated by reference in their entirety.

### Nucleic Acid Sequencing With Nanosensors Assimilating Polynucleotide Binding Proteins

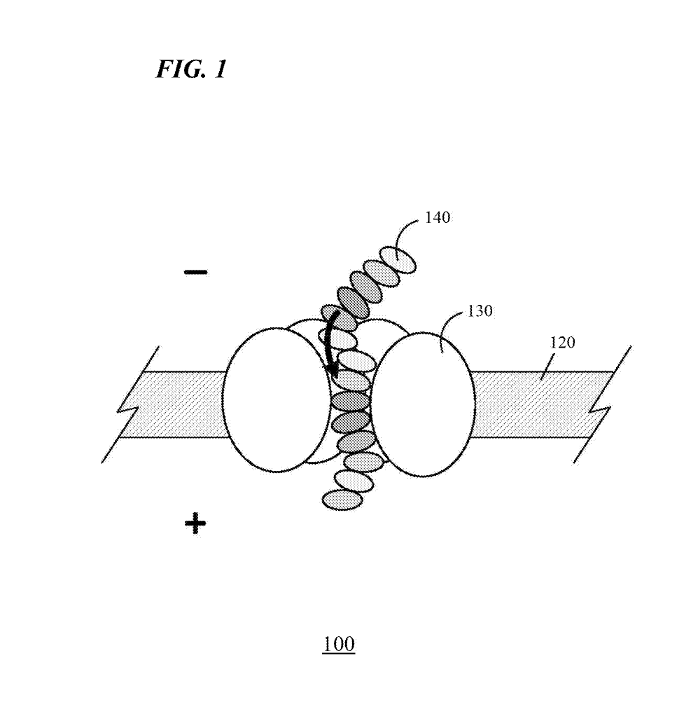

The invention provides systems, methods, and compositions for sequencing nucleic acids using nanosensors. In some embodiments, the invention described herein provides nanoscale biosensors, i.e., nanosensors that simplify the control and measurement of polynucleotides by adapting authentic polynucleotide binding proteins (herein abbreviated as “PBP”) for use as molecular sensor devices. According to the present invention, PBPs are assimilated with a membrane, or other structure or interface, to create an aperture for conductance when an electrical potential is applied across the membrane. At the same time, the PBP retains its natural DNA binding and, in some embodiments, nucleic acid processing activities. Such biosensors may be used for nucleic acid sequencing applications, as illustrated in FIG. 1. FIG. 1 depicts a generic nanosensor 100 in which PBP 130 is embedded in interface structure 120, which separates two reservoirs containing electrically conductive media. The interface structure is also referred to herein as a “high impedance support membrane”, or an “aperture support”. In this embodiment, the native quaternary structure of the PBP forms a central core, which creates the lowest impedance pathway for ions to pass between the reservoirs. In other words, the PBP forms an electroconductive pore in the membrane. As the core of the PBP forms an electroconductive pore, or ion channel, the PBP may also be regarded as a “nanopore”. A target polynucleotide 140 is bound by the PBP and, upon application of a voltage potential across interface 120, is translocated through the PBP core with a directionality denoted by the arrow. The rate of translocation is controlled by the natural polynucleotide binding, and, in some embodiments, modifying, or processing, activities of the PBP. As the individual nucleotide units comprising the polynucleotide sequentially pass through the constriction site, or zone, of the PBP core, each alters the ionic current in a characteristic manner. The polynucleotide thus spans the membrane (“membrane spanning”) as it translocates through the PBP. These nucleotide-specific alterations in ionic current are also referred herein to as “current modulation characteristics” or “current signatures”. Thus, each of the nucleotides in a polynucleotide template blocks the pore in a measurably different way, allowing for identification of bases in the strand, and thereby sequencing the polynucleotide.

Prior to the present disclosure, natural PBPs have not been fully considered as having the potential to form electrically conducting apertures, i.e. to function as molecular sensors, or nanopores. In contrast to PBPs, the transmembrane proteins used for nanopore sequencing methods described in the art (e.g., alpha-hemolysin and MspA), function naturally as exotoxins and, as such, are not designed by nature to interact with and process polynucleotides and to undergo conformational changes upon nucleic acid processing. As has been discussed, significant efforts have been made to engineer such transmembrane proteins in order to decrease their noise and enhance their base resolution characteristics. However, by utilizing the natural polynucleotide binding and processing functions of PBPs, the present invention provides improved molecular sensors which provide a structurally dynamic constriction site in an electroconductive pore that undergoes nucleotide dependent changes in conformation. These changes in conformation advantageously contribute to the signature ion current of each nucleotide. Although PBPs have been described in the art as components of nanopore sequencing assemblies (see, e.g. U.S. Patent Publication No. 2014/0051068 to Cherf et. al), prior to the present disclosure, they have not been recognized as having the potential to function as structurally dynamic transmembrane pores themselves and contribute to nucleotide-specific changes in ion current through the pore. The PBPs of the present invention may offer a higher degree of resolution with regard to both the composition and spatial relationship between nucleotide units within a target polynucleotide.

One exemplary class of PBPs contemplated by the present invention are DNA helicases. Helicases are a class of enzyme that function as motor proteins, moving directionally along a polynucleotide backbone while actively catalyzing strand duplex separation using the energy generated from nucleotide triphosphate (NTP) hydrolysis. DnaB-like helicases, such as bacteriophage T7 gp4, form a donut, or ring, -shaped quaternary structure, composed of a hexamer of protein subunits (see, e.g., Singleton, M. et al. (2000). Crystal Structure of T7 Gene 4 Ring Helicase Indicates a Mechanism for Sequential Hydrolysis of Nucleotides. Cell, 101, 589-600). T7gp4 translocates the leading 5′ nucleic acid strand through its active site core in a base-by-base manner, functioning as a dynamic molecular ratchet. Thus, the ring structure formed by DnaB-like helicases structure is well-suited for use as a molecular sensor, though the present invention is not intended to be so limited.

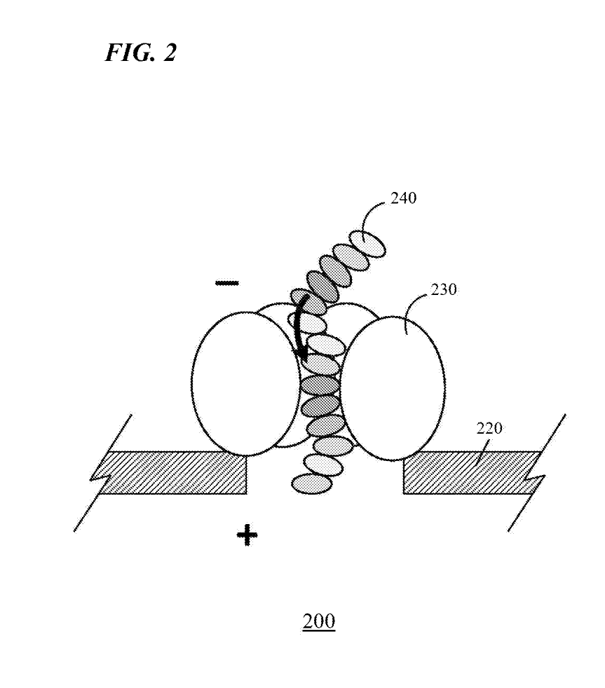

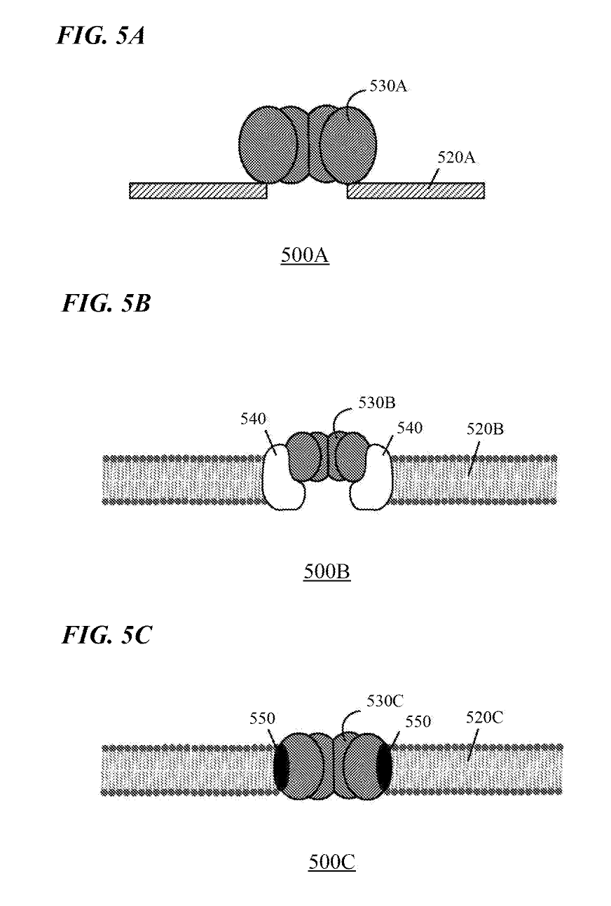

One embodiment of the present invention is illustrated in FIG. 2, which depicts the features of a generalized helicase-based molecular sensor 200, in which a helicase subunit assembly 230 is secured to a high impedance support membrane 220. For simplicity of illustration, the complete hexameric subunit assembly of the helicase is not shown. In this embodiment, the helicase is localized to an aperture preformed in the support membrane; however, the central channel (i.e. aperture) formed by the helicase subunit assembly presents the lowest impedance pathway for ion current to pass through the support membrane. In other words, the constriction site of the transmembrane pore is formed by the helicase protein itself Alternative means of assimilating PBPs with support membrane are also contemplated by the present invention, as discussed further below with reference to FIG. 5. A polynucleotide sequencing target 240 is complexed with the helicase such that the leading (5′ end) strand can be electrophoretically driven through the constriction site formed by the central core of the hexameric subunit assembly with a directionality denoted by the arrow. In this embodiment, ion current flowing through the constriction site formed in the helicase core is modulated by the polynucleotide and conformation of the helicase constriction site, and the sequence of the polynucleotide is obtained as described herein.

In one exemplary method of practicing the present invention, with continued reference to FIG. 2, support membrane 220 is used to separate two reservoirs that contain a buffered electrolytic reagent mix, i.e. an electrically conductive media (e.g., 1M KCl with 10 mM HEPES). Electrodes (e.g., Ag/AgCl) are placed in each reservoir to establish a voltage potential across the membrane, wherein the constriction site of the helicase provides the primary pathway for ions to pass between the reservoirs. The medium surrounding the helicase has the components required for helicase activity, including, when required, ATP or another suitable free nucleotide, and, optionally, an enzyme cofactor, e.g., a divalent cation, such as Mg²⁺. A double-stranded polynucleotide sequencing target, or template, is introduced and the helicase binds and unwinds the strands of the polynucleotide according to its inherent activities. The unwound polynucleotide strands are tensioned by the applied voltage and propagate through the helicase channel, while the concurrent ion conductance is measured. Changes in the ion current passing through the helicase channel result from blockages caused by individual nucleotides and the conformation of the constriction site as the template strands are processed. These changes in the ionic current, or current signatures, are then used to identify individual bases and obtain sequence information.

The nucleic acid sequencing methods of the present invention are possible because transmembrane pores formed by the PBPs can be used to differentiate nucleotides of similar structure on the basis of the different effects they have on the ion current passing through the pore. During the processing of a nucleotide in the target polynucleotide in the pore, the nucleotide affects the ion current flowing through the pore in a manner specific for that nucleotide. For example, a particular nucleotide will reduce the ion current flowing through the pore for a particular mean time period and to a particular extent. In other words, the ion current flowing through the pore is distinctive for a particular nucleotide. Control experiments may be carried out to determine the effect a particular nucleotide has on the ion current flowing through the pore. Results from carrying out the method of the invention on a test sample can then be compared with those derived from such a control experiment in order to determine the sequence of the target polynucleotide.

In contrast to conventional nanopore sequencing methods, which measure the electrical (impedance) characteristics of a string of polynucleotides as it translocates the limiting nanopore aperture, the methods of the present invention measure discreet, nucleotide-specific changes within the core of the PBP. In this manner, base-specific actions can be monitored in real-time as the target polynucleotide is processed by the PBP. These methods are unique in that they utilize the DNA handling functions of the PBP to modulate the geometry of the constriction site of the pore that are then directly reflected in ion current signals. Moreover, the constriction sites formed by the PBPs of the present invention are structurally dynamic and undergo base-specific conformational changes, which in some embodiments contribute to base-specific changes in conductance (i.e., current modulation characteristics). For example, in one embodiment, as a helicase PBP core binds and actively separates each base pair of the target polynucleotide, bulk changes in the geometry of the constriction site are induced by the specific nucleotides bound and consequent conformational changes within the helicase active site as the base pairs are separated. From these resulting ion current signatures, individual bases can be identified. Additionally, in other embodiments, the base-specific changes may be interpreted from ion current signatures generated before and after base pair separation. In yet other embodiments, a combined approach that utilizes ion current signatures before, during, and after base pair separation in conjunction with temporal (e.g., time between and duration of signal events) and noise characteristics can also be utilized to derive base-specific measurements.

The methods of the present invention contemplate several means to control helicase and other PBP activity. In certain embodiments, the activity of the PBP may be controlled through manipulating reaction conditions, e.g., temperature, salt concentration and composition, pH, sample viscosity, NTP concentration and type, enzyme cofactor type, and drive voltage to influence the polynucleotide binding and processing functions of the PBP.

For example, in some embodiments of the present invention reaction temperature may range from 0° C. to 100° C., from 15° C. to 95° C., from 16° C. to 90° C., from 17° C. to 85° C., from 18° C. to 80° C., from 19° C. to 70° C. or from 20° C. to 60° C. The methods are typically carried out at room temperature. The methods are optionally carried out at a temperature that supports enzyme function, such as about 37° C.

In some embodiments, the salt concentration may be low salt concentrations, for example less than 0.5M salt, or high salt concentrations, for example, at least about 0.5M, at least about 0.6M, at least about 1M, at least about 1.5M, at least about 2M, at least about 2.5M, at least about 3M, at least about 3.5M, at least about 4M, at least about 4.5M, at least about 5M, at least about 5.5M, and at saturation. Certain exemplary salts include, but are not limited, to any alkali metal chloride salt, e.g., potassium chloride (KCl), sodium chloride (NaCl) or caesium chloride (CsCl). KCl is typically preferred.

In other embodiments, the pH of the reaction may be adjusted to between about 6 and about 9. In some cases, the pH is between about 6.5 and about 8.0. In some cases, the pH is between about 6.5 and 7.5. In some cases, the pH is about 7.4.

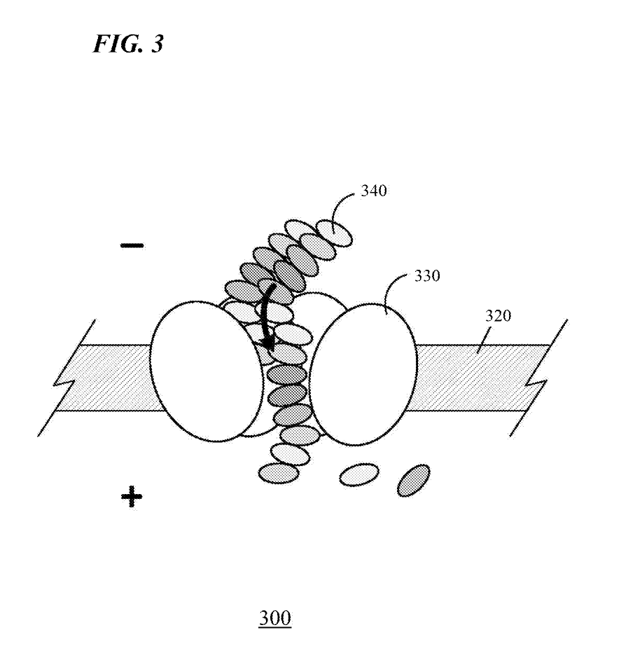

In other embodiments, certain exemplary NTPs include, but are not limited to ATP, CTP, GTP, and TTP. In other embodiments, the enzyme cofactor may be a divalent cation, e.g. Mg²⁺, Mn²⁺, Ca²⁺, or Co²⁺.

[48] FIG. 3 depicts another embodiment of the present invention in which the PBP 330 of molecular sensor 300 is an exonuclease enzyme. In some embodiments, the exonuclease may be, e.g., a phage lambda exonuclease. In the embodiment illustrated in FIG. 3, exonuclease 330 is secured, or assimilated, in a high impedance support membrane 320. In this configuration, the PBP spans the support membrane, although alternative means of assimilating PBPs into support membranes are also contemplated by the present invention, as discussed further below with reference to FIG. 5. As was discussed with reference to FIG. 2, the membrane is used to separate two reservoirs that contain a buffered electrolytic reagent mix (e.g., 1 M KCl with 10 mM HEPES). Electrodes (e.g. Ag/AgCl) are placed in each reservoir with an applied potential between them such that the central channel formed by the exonuclease provides the lowest impedance pathway for ions to pass between the reservoirs, i.e. forms the constriction point of the electroconductive pore. The medium surrounding the exonuclease contains the reagents necessary for exonuclease activity, including appropriate cofactors. As double-stranded target nucleotide 340 passes into the exonuclease, bases from the 5′ terminal end are cleaved off and diffuse away. The remaining DNA strand propagates through the enzyme channel, assisted by the applied voltage, and changes in the ion conductance are measured during this process. The volumetric changes in the channel resulting from the removal and diffusion of each base result in changes in the ionic current that are then measured for base identification. This process differs from that of transmembrane nanopores in that it utilizes the DNA processing functionality of the exonuclease to provide a controlled path for the DNA through the central channel and causes volumetric voids due to the cleaving of each base that, together with conformational changes in the enzyme during polynucleotide processing, can be translated into ionic signals.

In another embodiment of the present invention, the PBP of a molecular sensor is a DNA polymerase enzyme. DNA polymerases are made up of domains that move relative to one another during the polymerase reaction. The structure of a DNA polymerase is analogous to a right hand with a “finger” domain, a “palm” domain, and a “thumb” domain. Polymerases undergo conformational changes in the course of synthesizing a nucleic acid polymer. For example, polymerases undergo a conformational change from an open conformation to a closed conformation upon binding of a nucleotide. A polymerase that is bound to a nucleic acid template and growing primer with no free nucleotide present is in what is referred to in the art as an “open” conformation. When this polymerase complexes with a nucleotide that is the complement to the template base in the next extension position the polymerase reconfigures into what is referred to in the art as a “closed” conformation. At a more detailed structural level, the transition from the open to closed conformation is characterized by relative movement within the polymerase resulting in the “thumb” domain and “fingers” domain being closer to each other. In the open conformation the thumb domain is further from the fingers domain, akin to the opening and closing of the palm of a hand.

In particular embodiments, a DNA polymerase is assimilated with a membrane, as described in more detail with reference to FIG. 5, to form a transmembrane pore. The conformational movement of the polymerase can be used to distinguish the species of nucleotides that are added to a primed nucleic acid template during the polymerization reaction. For example, in the “open” configuration, the polymerase may be bound to a primed target, but not bound to an incoming nucleotide. In this “open” configuration, the constriction point formed by the polymerase may be substantially occluded and consequently will substantially restrict the flow of ion current through the pore during an applied potential. A second, e.g., “closed” configuration is induced, e.g., by binding of an incoming nucleotide to form a correct base pair with the template nucleic acid. In this second configuration, the degree to which the constriction point is occluded is reduced, and consequently the flow of current through the pore will increase. Both the conformational change of the polymerase and the specific nucleotide bound contribute to the modulation of ion current flow through the constriction point and generate an electronic signal specific for each nucleotide species. Electronic signals measured over time as the polymerase PBP synthesizes a daughter strand provides sequence information in real time based on the current modulation characteristics of each of the four individual nucleotides.

Suitable DNA polymerases for practice of the present invention include those described above; in some embodiments, the DNA polymerase is phi29 DNA polymerase. In other embodiments, the DNA polymerase may be VENT®(exo-) DNA polymerase, large (Klenow) fragment of E. coli DNA polymerase I, Bst polymerase, large fragment, Pfu DNA polymerase, KOD DNA polymerase, or TAQ DNA polymerase. It is preferable that the DNA polymerase have high processivity. The polymerases of the invention may, or may not, display strand displacement activity, depending on the particular application of interest. In addition, the polymerases of the invention may, or may not, display exonuclease activity, depending on the particular application of interest.

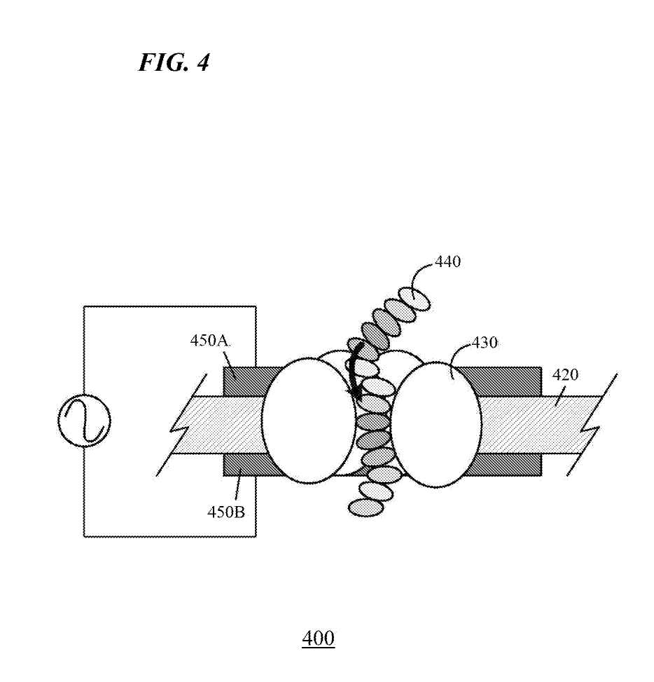

FIG. 4 depicts an alternative embodiment of the present invention in which nanosensor 400 assimilates a PBP 430 that is secured in a solid-state support, or membrane 420. In practice of this embodiment of the invention, rather than monitoring the ion current blockage, electrodes 450A and 450B are positioned on each side of the membrane, adjacent to the PBP. Base identities of target polynucleotide 440 are determined by monitoring the impedance changes in the protein/DNA/channel complex via fringe fields that emanate into this volume. As the PBP translocates the polynucleotide template in a base-by-base fashion, the entire complex reconfigures to accommodate the polynucleotide as it is bound and processed. The signature current modulations of these events are captured and reflect the changes in the impedance response to a high frequency drive potential. This detection technique can be adapted to accommodate a range of PBPs and nanosensors.

Several means for assimilating a PBP in a membrane with very high electrical impedance are contemplated by the present invention, certain embodiments of which are illustrated in FIGS. 5A-C. In one embodiment, as illustrated in FIG. 5A, PBP 530A is localized to a pore, or hole, formed in a solid-state membrane 520A. As discussed herein, it is obligatory to the present invention that the resistance of the pore or hole be much lower than that of the PBP so that the variation in the measured ion conductance can be attributed to the nucleotides that are complexed within the PBP active site and not due to nucleotides that may be positioned in the support aperture. In certain embodiments, arrays of nano-scale holes with diameters of, e.g., 4 nm can be efficiently drilled on a large scale by a processing technique utilizing the Zeiss Helium Ion microscope (see, e.g., Yang, J. et al. (2011). Helium Ion Microscope Fabrication of Solid-State Nanopores for Biomolecule Detection. Zeiss Application Note).

In another embodiment, as illustrated in FIG. 5B, PBP 530B is localized to the large aperture formed by a natural pore forming protein 540 embedded in a lipid bilayer 520B. In this embodiment, the natural pore forming protein functions as a support pore for the PBP and the PBP provides the constriction point of the transmembrane pore formed by the protein assembly. One exemplary pore forming protein is the phi 29 connector, a transmembrane protein with a pore diameter of ˜3.9 nm (see, e.g., Geng, J. et al., (2011). Three Reversible and Controllable Discrete Steps of Channel Gating of a Viral DNA Packaging Motor. Biomaterials. 32(32), 8234-8242). Other exemplary support pores are the ClyA pore (see, e.g. Franceschini, L. et al. A nanopore machine promotes the vectorial transport of DNA across membranes. Nat. Commun. 4:2415 doi: 10.1038/ncomms3415. 2013), the FhuA pore (see, e.g. Mohammad, M. et al. Redesign of a Plugged β-Barrel Membrane Protein. Journal Biol. Chem. 286.10. 2011: 8000-8013), and the MscL pore (see, e.g., Cruickshank, C. et al. Estimation of the Pore Size of the Large-Conductance Mechanosensitive Ion Channel of Escherichia coli. Biophysical J. 73:1925-1931. 1997).

In some embodiments, the PBPs can be directed to the pores by attachment of charged polymeric leaders, e.g., in a manner that has been demonstrated by Hall and colleagues (see, e.g., Hall, A. et al. (2010). Hybrid Pore Formation by Directed Insertion of α-Haemolysin into Solid-State Nanopores. Nat. Nanotech. 5(12), 874-877). The seal that results from positioning the PBP in the membrane hole may be maintained by the electrophoretic force acting on the target polynucleotide, but in some embodiments, may also be promoted by covalent or noncovalent bonds engineered at the membrane/PBP interface.

In yet another embodiment, as depicted in FIG. 5C, PBP 530C is assimilated with a membrane by embedding the PBP directly into a lipid bilayer membrane 520C. This configuration may be facilitated, e.g., by modifications of the PBP protein itself that introduces hydrophobic groups 550 on the outer surface of the PBP, thereby guiding incorporation of the PBP into the bilayer. Such modifications may be introduced through artificially engineering the protein according to molecular biological methods well known in the art, as summarized below.

Nucleic acids encoding the PBPs can be obtained using routine techniques in the field of recombinant genetics. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994-1999). Such nucleic acids may also be obtained through in vitro amplification methods such as those described herein and in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117, each of which is incorporated by reference in its entirety for all purposes and in particular for all teachings related to amplification methods.

Modifications can additionally be made to the PBP without diminishing its biological activity. Some modifications may be made to facilitate the cloning, expression, or incorporation of a domain into a protein. Such modifications can include, for example, the addition of codons at either terminus of the polynucleotide that encodes the binding domain to provide, for example, a methionine added at the amino terminus to provide an initiation site, or additional amino acids (e.g., poly His) placed on either terminus to create conveniently located restriction sites or termination codons or purification sequences.

The modified PBP described herein can be expressed in a variety of host cells, including E. coli, other bacterial hosts, yeasts, filamentous fungi, and various higher eukaryotic cells such as the COS, CHO and HeLa cells lines and myeloma cell lines. Techniques for gene expression in microorganisms are described in, for example, Smith, Gene Expression in Recombinant Microorganisms (Bioprocess Technology, Vol. 22), Marcel Dekker, 1994.

There are many expression systems for producing the modified PBPs described herein that are known to those of ordinary skill in the art. See, e.g., Gene Expression Systems, Fernandex and Hoeffler, Eds. Academic Press, 1999; Sambrook and Russell, supra; and Ausubel et al, supra.) Typically, the polynucleotide that encodes the fusion polypeptide is placed under the control of a promoter that is functional in the desired host cell. Many different promoters are available and known to one of skill in the art, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Accordingly, the nucleic acids that encode the joined polypeptides are incorporated for high level expression in a desired host cell.

Expression control sequences that are suitable for use in a particular host cell are often obtained by cloning a gene that is expressed in that cell. Commonly used prokaryotic control sequences, which are defined herein to include promoters for transcription initiation, optionally with an operator, along with ribosome binding site sequences, include such commonly used promoters as the beta-lactamase (penicillinase) and lactose (lac) promoter systems (Change et al., Nature (1977) 198: 1056), the tryptophan (trp) promoter system (Goeddel et al., Nucleic Acids Res. (1980) .delta.: 4057), the tac promoter (DeBoer, et al., Proc. Natl. Acad. Sci. U.S.A. (1983) 80:21-25); and the lambda-derived PL promoter and N-gene ribosome binding site (Shimatake et al., Nature (1981) 292: 128). The particular promoter system is not critical, any available promoter that functions in prokaryotes can be used. Standard bacterial expression vectors include plasmids such as pBR322-based plasmids, e.g., pBLUESCRIPT™, pSKF, pET23D, lambda-phage derived vectors, and fusion expression systems such as GST and LacZ. Epitope tags can also be added to recombinant proteins to provide convenient methods of isolation, e.g., c-myc, HA-tag, 6-His tag, maltose binding protein, VSV-G tag, anti-DYKDDDDK tag, or any such tag, a large number of which are well known to those of skill in the art.

A variety of protein isolation and detection methods are known and can be used to isolate enzymes, e.g., from recombinant cultures of cells expressing the recombinant enzymes of the invention. A variety of protein isolation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997); Bioseparation of Proteins, Academic Press, Inc.; Bollag et al. (1996), Satinder Ahuja ed., Handbook of Bioseparations, Academic Press (2000).

An alternative detection methodology contemplated by the present invention, and applicable to all embodiments disclosed herein, is based on optical signals, and is disclosed in published PCT application no. WO/2010/088557, entitled, “High Throughput Nucleic Acid Sequencing by Expansion and Related Methods”, herein incorporated by reference in its entirety. In brief, an optically detectable agent is introduced into the cis reservoir of a nanosensor system. The agent is capable of flowing into the trans reservoir by passing through the channel formed by the PBP. Thus, the concentration of the agent in the trans reservoir is controlled by the PBP. The concentration of the agent in the trans reservoir may be further modulated by the coincident passage of a polynucleotide through the PBP. In practice, the modulation of the agent's concentration is measured optically, either directly or indirectly, and the resulting measurements are correlated to specific nucleotides to obtain sequence information related to the polynucleotide. One exemplary optically detectable agent contemplated by the present invention is fluorescein, which can be monitored with any suitable excitation light known in the art. Another exemplary agent is the divalent ion calcium, which is detected when bound to a second, indicator, reagent introduced to the trans reservoir. In certain embodiments suitable calcium indicators contemplated by the present invention include, but are not limited to fluorescent probes, e.g., Fluo-3, Fluo-4, and Fluo-5, available from Molecular Probes (Invitrogen).

The target, or template, polynucleotides of the present invention are present in any suitable sample. The invention is typically carried out on a sample that is known to contain or suspected to contain the target polynucleotide. Alternatively, the invention may be carried out on a sample to confirm the identity of one or more target polynucleotides whose presence in the sample is known or expected.

The sample may be a biological sample. The invention may be carried out in vitro on a sample obtained from or extracted from any organism or microorganism. The organism or microorganism is typically prokaryotic or eukaryotic and typically belongs to one the five kingdoms: plantae, animalia, fungi, monera and protista. The invention may be carried out in vitro on a sample obtained from or extracted from any virus. The sample is preferably a fluid sample. The sample typically comprises a body fluid of the patient. The sample may be urine, lymph, saliva, mucus or amniotic fluid but is preferably blood, plasma or serum. Typically, the sample is human in origin, but alternatively it may be from another mammal animal such as from commercially farmed animals such as horses, cattle, sheep or pigs or may alternatively be pets such as cats or dogs. Alternatively a sample of plant origin is typically obtained from a commercial crop, such as a cereal, legume, fruit or vegetable, for example wheat, barley, oats. canola, maize, soya, rice, bananas, apples, tomatoes, potatoes, grapes, tobacco, beans, lentils, sugar cane, cocoa, cotton.

The sample may be a non-biological sample. The non-biological sample is preferably a fluid sample. Examples of a non-biological sample include surgical fluids, water such as drinking water, seawater or river water, and reagents for laboratory tests.

The sample is typically processed prior to being assayed, for example by centrifugation or by passage through a matrix that filters out unwanted molecules or cells, such as red blood cells. Nucleic acids are typically further purified by any of the methods known in the art, e.g., those based on phenol-chloroform extraction, differential precipitation, ethanol precipitation, or in-gel separation. Sample preparation methods may be performed with commercially available kits, often based on solid-phase separation, such as those provided by QIAGEN. The sample may be measured immediately upon being taken. The sample may also be typically stored prior to assay, preferably below −70° C.

Any membrane support aperture may be used in accordance with the invention. Suitable membranes are well-known in the art. In some embodiments, the membrane is an amphiphilic layer. An amphiphilic layer is a layer formed from amphiphilic molecules, such as phospholipids, which have both hydrophilic and lipophilic properties. Preferred phospholipids of the present invention include 1,2-diphytanoyl-sn-glycero-3-phosphoethanolamine (PE); 1,2-diphytanoyl-sn-glycero-3-phosphocholine (PC), and 1,2-diphytanoyl-sn-glycero-3-phospho-(1′-rac-glycerol) (PG).

In other embodiments, the membrane is a solid state layer. A solid-state layer is not of biological origin. In other words, a solid state layer is not derived from or isolated from a biological environment such as an organism or cell, or a synthetically manufactured version of a biologically available structure. Solid state layers can be formed from both organic and inorganic materials including, but not limited to, microelectronic materials, insulating materials such as Si3N4, Al203, and SiO, organic and inorganic polymers such as polyamide, plastics such as Teflon® or elastomers such as two-component addition-cure silicone rubber, and glasses. The solid state layer may be formed from graphene. Suitable solid-state films contemplated by the present invention include, but are not limited to, silicon nitride, silicon dioxide, silicon carbide, graphene, and other metal oxides (e.g., aluminum oxide and titanium oxide). Such thin, solid-state film have demonstrated gigaohm resistances. In some embodiments, organic coatings, such as silanes, SAMs, and lipid layers may be used to further insulate the film and provide additional surface functionality, such as reduction of non-specific fouling. In other embodiments, the membrane of the nanosensors of the present invention may be an organic membrane, including, but not limited to, polymers and lipid bilayers. These have also demonstrated gigaohm resistances. In some embodiments, sealing methods are employed to limit ions that “leak” between the membrane and the PBP, thereby reducing a source of background noise on the ion current signal. The solid state layer may further comprise a solid state pore or a plurality of such pores. The solid state layer or pore may further comprise a linker group compound that is attached by covalent bond. A PBP may be attached to a solid state layer or solid state pore using a suitable linker group.

The invention is generally described by reference to a single PBP, but the invention anticipates using arrays of PBPs from, e.g., around 10 PBPs to around 10 million PBPs. In some cases, arrays of around 10 PBPs to around 1000 PBPs are used. In some cases, arrays of around 100 PBPs to around 10,000 PBPs are used. In other cases, arrays of PBPs from around 1,000 PBPs to around 1 million PBPs are used.

### Apparatus and Systems

The methods of the invention may be carried out using any apparatus that is suitable for investigating a nanosensor complex comprising a polynucleotide binding protein of the invention assimilated with a membrane. The methods may be carried out using any apparatus that is suitable for stochastic sensing. For example, an apparatus comprising a chamber comprising an aqueous solution and a barrier that separates the chamber into two sections. The barrier may have an aperture in which the membrane containing the complex is formed. The nucleotide or nucleic acid may be contacted with the complex by introducing the nucleic acid into the chamber. The nucleic acid may be introduced into either of the two sections of the chamber, but is preferably introduced into the section of the chamber containing the PBP.

The methods involve measuring the ion current passing through the pore during PBP handling of the target nucleic acid. Therefore the apparatus also comprises an electrical circuit capable of applying a potential and measuring an electrical signal across the membrane and pore. The methods may be carried out using a patch clamp or a voltage clamp. The method preferably involves the use of a voltage clamp.

The methods of the invention involve the measuring of an ion current passing through the pore during PBP handling of the target nucleic acid. Suitable conditions for measuring ionic currents through transmembrane protein pores are known in the art and disclosed herein. The method is carried out with a voltage applied across the membrane and pore, also referred to herein as a “voltage drop”. The voltage used is typically from −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 120 mV to 170 mV. It is possible to increase discrimination between different nucleotides processed by a complex of the invention by using an increased applied potential.

The methods are carried out in the presence of any alkali metal chloride, acetate, or mixture of chloride and acetate salt. In the exemplary apparatus discussed above, the salt is present in the aqueous solution in the chamber. Potassium chloride (KCl), sodium chloride (NaCl) or ammonium chloride (NH₄Cl) is typically used. KCl or NH₄Cl is preferred. The salt concentration is typically from 0.1 to 2.5M, from 0.3 to 1.9M, from 0.5 to 1.8M, from 0.7 to 1.7M, from 0.9 to 1.6M or from 1M to 1.4M. High salt concentrations provide a high signal to noise ratio and allow for ion currents indicative of the presence of a nucleotide to be identified against the background of normal ionic current fluctuations. However, lower salt concentrations are preferably used so that the enzyme is capable of functioning. The salt concentration is preferably from 150 to 500 mM. Good signal distinction at these low salt concentrations can be achieved by carrying out the method at temperatures above room temperature, such as from 30° C. to 40° C.

In addition to increasing the solution temperature, there are a number of other strategies that can be employed to increase the conductance of the solution, while maintaining conditions that are suitable for PBP activity. One such strategy is to use the lipid bilayer to divide two different concentrations of salt solution, a low salt concentration of salt on the enzyme side and a higher concentration on the opposite side.

The invention relates in some aspects to systems for sequencing with polynucleotide binding proteins. In some cases, the systems comprise devices with resistive openings between fluid regions in contact with the sensor complex and fluid regions which house a drive electrode. The devices of the invention can be made using a semiconductor substrate such as silicon to allow for incorporated electronic circuitry to be located near each pore of a complex. The devices of the invention will therefore comprise arrays of both microfluidic and electronic elements. In some cases, the semiconductor which has the electronic elements also includes microfluidic elements that contain the sensor complexes. In some cases, the semiconductor having the electronic elements is bonded to another layer which has incorporated microfluidic elements that contain the sensor complexes.

The devices of the invention generally comprise a microfluidic element into which a PBP is disposed. This microfluidic element will generally provide for fluid regions on either side of the sensor complex through which the ion current to be detected for sequence determination will pass as described above. In some cases, the fluid regions on either side of the sensor complex are referred to as the cis and trans regions, where ion current generally travels from the cis region to the trans region through the pore. For the purposes of description, the terms upper and lower are also used to describe such reservoirs and other fluid regions. It is to be understood that the terms upper and lower are used as relative rather than absolute terms, and in some cases, the upper and lower regions may be in the same plane of the device. The upper and lower fluidic regions are electrically connected either by direct contact, or by fluidic (ionic) contact with drive and measurement electrodes. In some cases, the upper and lower fluid regions extend through a substrate, in other cases, the upper and lower fluid regions are disposed within a layer, for example, where both the upper and lower fluidic regions open to the same surface of a substrate. Methods for semiconductor and microfluidic fabrication described herein and as known in the art can be employed to fabricate the devices of the invention.

The invention involves the use of an electrode to sense potential in a fluidic region. The electrode may be made of any suitable material. The electrode generally comprises a conductor or a semiconductor. For example, the electrode can be a metal, a semiconductive metal oxide, or a semiconductor such as silicon or gallium arsenide. In some cases the electrode is coated with a thin insulating layer that allows for the electrode to sense the potential without being directly exposed to the fluid. The insulating layer can comprise an inorganic or organic material. The insulating layer can be deposited, plated, or grown onto the electrode surface, for example by chemical vapor deposition. In some cases the electrode that senses the potential comprises a component in an electrical circuit. For example, the electrode can comprise the gate of a transistor including the gate of a naked transistor. The electrode is generally connected to or is part of an electronic component that is used to measure the potential. In some cases the component is a transistor or series of transistors. The electronic component can also comprise a capacitor or other suitable component. In some cases the electrode comprises a conductor (e.g. a wire) that is in contact with the solution (with or without an insulating layer), which extends from the fluid to an electronic component for measuring potential. This electronic component can be in the substrate that is in contact with the fluid, or the conductor can extend to an electronic component off of the substrate. In preferred embodiments, the electrode is in direct contact with or comprises a portion of an electrical component on the substrate. Such electrical component can be, for example, a transistor.

Systems of the invention may include a computer, which may implement, control, and/or regulate the voltage of a voltage source, measurements of an ammeter, and display of the ionic current graphs as discussed herein.

Various methods, procedures, circuits, elements, and techniques discussed herein may also incorporate and/or utilize the capabilities of a computer. Moreover, capabilities of a computer may be utilized to implement features of exemplary embodiments discussed herein. One or more of the capabilities of the computer may be utilized to implement, to connect to, and/or to support any element discussed herein (as understood by one skilled in the art) and in FIGS. 1 and 2. For example, the computer may be any type of computing device and/or test equipment (including ammeters, voltage sources, connectors, etc.). An input/output device (having proper software and hardware) of a computer may include and/or be coupled to the molecular sensor complex apparatus discussed herein via cables, plugs, wires, electrodes, patch clamps, etc. Also, the communication interface of the input/output devices comprises hardware and software for communicating with, operatively connecting to, reading, and/or controlling voltage sources, ammeters, and current traces (e.g., magnitude and time duration of current), etc., as discussed herein. The user interfaces of the input/output device may include, e.g., a track ball, mouse, pointing device, keyboard, touch screen, etc., for interacting with the computer, such as inputting information, making selections, independently controlling different voltages sources, and/or displaying, viewing and recording current traces for each base, molecule, biomolecules, etc.

While the disclosed subject matter is described herein in terms of certain embodiments, those skilled in the art will recognize that various modifications and improvements can be made to the application without departing from the scope thereof. Thus, it is intended that the present application include modifications and variations that are within the scope of the appended claims and their equivalents. Moreover, although individual features of one embodiment of the application can be discussed herein or shown in the drawings of one embodiment and not in other embodiments, it should be apparent that individual features of one embodiment can be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the application such that the application should be recognized as also specifically directed to other embodiments having any other possible combinations. Thus, the foregoing description of specific embodiments of the application has been presented for purposes of illustration and description. It is not intended to be exhaustive or to limit the application to those embodiments disclosed.

### EXAMPLES

### Example 1

### ASSEMBLY OF THE CLYA TRANSMEMBRANE PORE WITH A LIPID BILAYER MEMBRANE TO FORM A SUPPORT PORE FOR A POLYNUCLEOTIDE BINDING PROTEIN

This Example demonstrates how a transmembrane pore protein may be assembled with a lipid bilayer membrane to provide a support pore for a PBP. The lipid bilayer membrane is formed with a phospholipid that exhibits high mechanical and chemical stability and has high electrical resistance. In this Example, the lipid is 1,2-diphytanoyl-sn-glycero-3-phosphoethanolamine (C₄₅H₉₀NO₈P), with a molecular weight of 804.172. Briefly, lipid bilayers are formed over an aperture in a PTFE solid support cell by first priming the cell with a thin coat of lipid. In this case, 1,2-diphytanoyl-sn-glycero-3-phosphoethanolamine (PE) is dissolved in hexane and coated over the support cell. Then, the hexane is removed by air-drying the painted cell. Next, the lipid is painted over the support cell by dissolving PE in 1-hexadecene and depositing the solution over the primed support cell with a pipette and moving an air bubble over the aperture in the support cell to form a lipid bilayer membrane over the aperture.

The transmembrane pore protein that functions as a support pore is a protein that naturally forms a large diameter transmembrane pore. In this Example, the transmembrane pore protein is ClyA, which is has advantageously been reported to display low intrinsic noise in planar lipid bilayer recordings. The geometry of the ClyA pore is suitable for providing a support pore for a PBP of the present invention, with a length of ˜13.0 nm, an aperture with an upper opening of diameter ˜6.4 nm and a lower opening of ˜3.3 nm, and a lumen of ˜5.5 nm (see, e.g., Franceschini, L. et al. A nanopore machine promotes the vectorial transport of DNA across membranes. Nat. Commun. 4:2415 doi: 10.1038/ncomms3415 (2013). The ClyA protein is assembled with the PE bilayer membrane using methods well known in the art, e.g., adding a solution of the protein to the lipid bilayer and allowing the protein to self-assemble with the membrane to form a transmembrane support pore.

A test apparatus has 2 reservoirs filled with electrolyte solution which are separated by the lipid membrane so that the only fluid connection between the reservoirs is through a pore. Each reservoir has a Ag/AgCl electrode through which potential is applied and current can be measured with a Molecular Devices Axopatch 200B amplifier.

### Example 2

### ASSEMBLY AND USE OF A NANOSENSOR COMPLEX COMPOSED OF A DNA POLYMERASE POLYNUCLEOTIDE BINDING PROTEIN AND A CLYA SUPPORT PORE

This Example describes how a DNA polymerase PBP may be assimilated with a support pore embedded in a lipid bilayer membrane to form a nanosensor complex for DNA sequencing applications. In this Example, the PBP is the Phi29 DNA polymerase with inherent polynucleotide strand-displacement and exonuclease activities. First, a lipid bilayer membrane is formed with the lipid 1,2-diphytanoyl-sn-glycero-3-phosphoethanolamine (C₄₅H₉₀NO₈P). Briefly, as described previously, the lipid bilayer is formed over an aperture in a PTFE solid support cell by priming the cell with a thin coat of lipid dissolved in hexane and coating over the support cell. Hexane is removed by air-drying the painted cell and lipid is painted over the support cell by dissolving PE in 1-hexadecene and depositing the solution over the primed support cell with a pipette and moving an air bubble over the aperture in the support cell to form a lipid bilayer membrane over the aperture. Next, an aqueous solution of the transmembrane pore protein, ClyA, is added to the lipid bilayer and the pore is allowed to self-assemble on the membrane and insert to form a transmembrane support pore.

A PBP-DNA template complex is generated next. In this Example, the Phi29 DNA polymerase PBP, double-stranded DNA template, and oligonucleotide primers are produced using standard molecular biology technologies. The double-stranded DNA template is complexed with an appropriate oligonucleotide primer and the primed DNA template is incubated with the Phi29 DNA polymerase in an aqueous solution containing 30 mM Tris-HCl, pH 7.5, 10 mM MgCl2, 4 mM Dithiothreitol, and 30 mM Ammonium Acetate, which binds the complex following its natural functions. The Phi29 polymerase-DNA template assembly is then assimilated, or coupled, with the ClyA support pore embedded in the lipid bilayer membrane by adding the assembly to the cis reservoir of the nanopore sensor containing an aqueous solution of 30 mM Tris-HCl, pH 7.5, 10 mM MgCl2, 4 mM Dithiothreitol, and 300 mM Ammonium Acetate in the cis reservoir and an aqueous solution of 1000 mM NH4Cl on the trans side reservoir and applying an electric potential across the membrane to thread the negatively charged DNA template molecule through the pore, thereby guiding and anchoring the DNA polymerase into the support pore.

While maintaining a positive trans side voltage bias, sequencing of the DNA template with the Phi29 DNA polymerase-ClyA support pore nanosensor is initiated by adding a mixture of all 4 deoxynucleotide triphosphate substrates to the cis side reservoir to a final concentration of 100 uM of each dNTP. Temperature of the sensor is maintained at 23° C. during the sequencing reaction. A voltage of 80 mV is applied and maintained across the membrane while conductivity through the membrane is measured over time as the polymerase processes the template nucleic acid according to its natural functions.

### Example 3

### ASSEMBLY AND USE OF A NANOSENSOR COMPLEX COMPOSED OF A DNA EXONUCLEASE POLYNUCLEOTIDE BINDING PROTEIN AND A CLYA SUPPORT PORE

This Example describes how a DNA exonuclease PBP may be assimilated with a support pore embedded in a lipid bilayer membrane to form a nanosensor complex for DNA sequencing applications. In this Example, the PBP is the phage lamda DNA exonuclease with inherent 5′ to 3′ exonuclease activities. First, a lipid bilayer membrane is formed with the lipid 1,2-diphytanoyl-sn-glycero-3-phosphoethanolamine (C₄₅H₉₀NO₈P). Briefly, as described previously, the lipid bilayer is formed across an aperture in a PTFE solid support cell by priming the cell with a thin coat of lipid dissolved in hexane and coating over the support cell. Hexane is removed by air-drying the painted cell and a lipid membrane is painted over the support cell by dissolving PE in 1-hexadecene and depositing the solution over the primed support cell with a pipette and moving an air bubble over the aperture in the support cell to form a lipid bilayer membrane over the aperture. Next, an aqueous solution of the transmembrane pore protein, ClyA, is added to the lipid bilayer and the pore is allowed to self-assemble on the membrane and insert to form a transmembrane support pore.

A PBP-DNA template complex is next generated. In this Example, the phage lambda DNA exonuclease PBP and double-stranded DNA template are produced using standard molecular biology technologies. The 5′ ends of the double-stranded DNA template are phosphorylated using well-known T4 Polynucleotide Kinase based methods. The resulting modified template is purified to using well-known silica glass fiber methods. The double-stranded DNA template is incubated with the phage lambda DNA exonuclease in an aqueous solution containing 30 mM Tris-HCl, pH 7.5, 2 mM EDTA, 4 mM DTT, and 30 mM ammonium acetate which binds the DNA template following its natural functions but does not initiate exonuclease digestion due to the lack of magnesium cofactor. The lambda exonuclease-DNA template assembly is then assimilated, or coupled, with the ClyA support pore embedded in the lipid bilayer membrane by adding the assembly to the cis reservoir of the nanopore sensor containing an aqueous solution of 30 mM Tris-HCl, pH 7.5, 2 mM EDTA, 4 mM Dithiothreitol, and 300 mM Ammonium Acetate in the cis reservoir and an aqueous solution of 1000 mM NH4Cl on the trans side reservoir and applying an electric potential across the membrane to thread the negatively charged DNA template molecule through the pore, thereby guiding and anchoring the DNA exonuclease complex into the support pore.

While maintaining a positive trans side voltage bias, sequencing of the DNA template with the lambda DNA exonuclease-ClyA support pore nanosensor is initiated by adding MgCl2, a cofactor necessary for exonuclease activity, to a final concentration of 10 mM in the cis reservoir. Temperature is maintained at 23° C. during the sequencing reaction. A voltage of 80 mV is applied and maintained and conductivity through the membrane is measured over time as the exonuclease processes the template nucleic acid according to its natural functions.

### Example 4

### ASSEMBLY AND USE OF A NANOSENSOR COMPLEX COMPOSED OF A DNA HELICASE POLYNUCLEOTIDE BINDING PROTEIN AND A CLYA SUPPORT PORE

This Example describes how a DNA helicase PBP may be assimilated with a support pore embedded in a lipid bilayer membrane to form a nanosensor complex for DNA sequencing applications. In this Example, the PBP is the DnaB-like helicase, bacteriophage T7 gp4, with inherent duplex strand separation activity. First, a lipid bilayer membrane is formed with the lipid 1,2-diphytanoyl-sn-glycero-3-phosphoethanolamine (C₄₅H₉₀NO₈P). Briefly, as described previously, the lipid bilayer is formed over an aperture in a PTFE solid support cell by priming the cell with a thin coat of lipid dissolved in hexane and coating over the support cell. Hexane is removed by air-drying the painted cell and a lipid membrane is painted over the support cell by dissolving PE in 1-hexadecene and depositing the solution over the primed support cell with a pipette and moving an air bubble over the aperture in the support cell to form a lipid bilayer membrane over the aperture. Next, an aqueous solution of the transmembrane pore protein, ClyA, is added to the lipid bilayer and the pore is allowed to self-assemble in the membrane and insert to form a transmembrane support pore.

A PBP-DNA template complex is next generated. In this Example, the DNA helicase PBP and double-stranded DNA template are produced using standard molecular biology technologies. The double-stranded DNA template is incubated with the T7 gp4 DNA helicase in an aqueous solution containing 30 mM Tris-HCl, pH 7.5, 10 mM MgCl₂ 4 mM DTT, and 30 mM ammonium acetate, which binds the DNA template following its natural functions. The T7 gp4 helicase-DNA template assembly is then assimilated, or coupled, with the ClyA support pore embedded in the lipid bilayer membrane by adding the assembly to the cis reservoir of the nanopore sensor containing an aqueous solution of 30 mM Tris-HCl, pH 7.5, 10 mM MgCl2, 4 mM Dithiothreitol, and 300 mM Ammonium Acetate in the cis reservoir and an aqueous solution of 1000 mM NH4Cl on the trans side reservoir and applying an electric potential across the membrane to thread the negatively charged DNA template molecule through the pore, thereby guiding and anchoring the DNA exonuclease into the support pore.

While maintaining a positive trans side voltage bias, sequencing of the DNA template with the T7 gp4 DNA helicase-ClyA support pore nanosensor is initiated by adding ATP to the cis reservoir. Temperature is maintained at 23° C. during the sequencing reaction. A voltage of 80 mV is applied and maintained and conductivity through the membrane is measured over time as the helicase processes the template nucleic acid according to its natural functions.

### Example 5

### ASSEMBLY AND USE OF A LOW-NOISE SOLID-STATE NANOSENSOR COMPLEX COMPOSED OF A DNA POLYMERASE POLYNUCLEOTIDE BINDING PROTEIN AND A SOLID-STATE CHIP

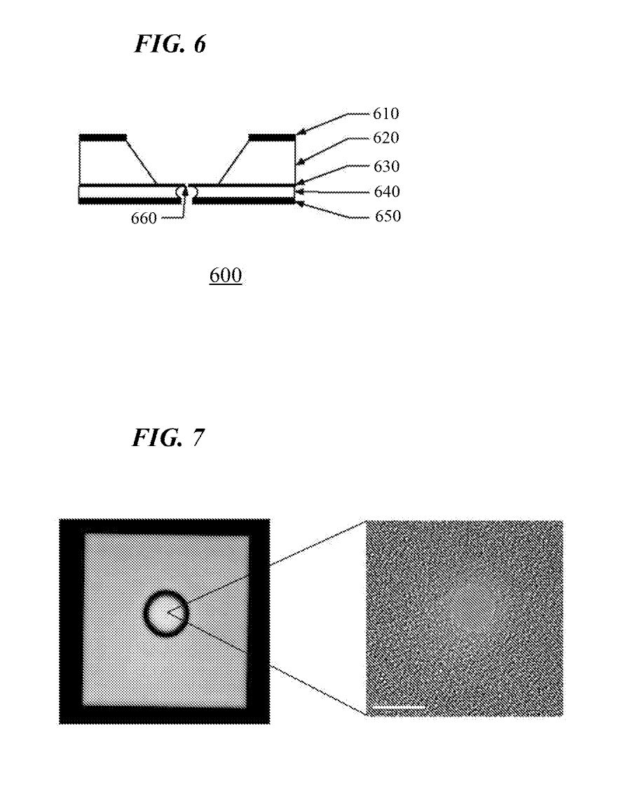

This Example describes how a DNA polymerase PBP may be assimilated with a low-noise solid-state support chip to form a nanosensor complex for DNA sequencing applications. In this Example, the PBP is the Phi29 DNA polymerase with inherent polynucleotide strand-displacement and exonuclease activities. First, a low capacitive solid-state chip is fabricated starting from a silicon chip with dimensions of 200 μm×10 μm. The chip is cleaned using the RCA process and then the following coatings are applied to the chip: 1) 30 nm LPCVP silicon (Si) lean silicon nitride (SiN) on both sides; 2) 3 μm PECVD SiO₂ on the backside of the chip; 3) 200 nm PECVD SiN on the backside of the chip. Lithography masking technology is then used to ME etch wells of 30 nm into the SiN on the frontside of the chip. Lithography masking technology is then further used to RIE etch wells of 200 nm on the on the backside of the chip. Finally, KOH aniso/isotropic etching is used to create the geometry of the solid state sensor chip 600 as depicted in FIG. 6, which illustrates SiN mask 610, Si substrate 620, SiN membrane 630, SiO2 layer 640, and SiN mask 650. The support pore 660, 4 nm in diameter, is drilled into the 30 nm thick silicon nitride membrane as denoted by the arrow using a FEI Technai-transmission electron microscope. FIG. 7 depicts an electron micrographic image of an electroconductive pore drilled into a solid-state silicon chip according to the methods of the present invention.

A test apparatus has two reservoirs filled with electrolyte solution which are separated by the silicon chip mounted on a gasket so that the only fluid connection between the reservoirs is through pore 660 located in the silicon nitride membrane of the chip. Each reservoir has a Ag/AgCl electrode through which potential is applied and current can be measured with a Molecular Devices Axopatch 200B amplifier.

A PBP-DNA template complex is generated next. In this Example, the Phi29 DNA polymerase PBP, double-stranded DNA template, and oligonucleotide primers are produced using standard molecular biology technologies. The double-stranded DNA template is complexed with an appropriate oligonucleotide primer and the primed DNA template is incubated with the Phi29 DNA polymerase in an aqueous solution containing 30 mM Tris-HCl, pH 7.5, 10 mM MgCl₂ 4 mM DTT, and 30 mM ammonium acetate, which binds the complex following its natural functions. The Phi29 polymerase-DNA template assembly is then assimilated, or coupled, with the solid-state chip by adding the polymerase assembly to the cis reservoir of the test apparatus containing an aqueous solution of 30 mM Tris-HCl, pH 7.5, 10 mM MgCl2, 4 mM Dithiothreitol, and 300 mM Ammonium Acetate in the cis reservoir and an aqueous solution of 1000 mM NH4Cl on the trans side reservoir and applying a positive electrical potential to the trans reservoir. The negatively charged DNA template molecule will then thread through the pore, thereby guiding and anchoring the DNA polymerase into the chip, depicted in FIG. 6 by arrow 670.

While maintaining a positive trans side voltage bias, sequencing of the DNA template with the solid-state nanosensor chip is initiated by adding a mixture of all four deoxyribonucleotide triphosphate substrates to the cis side of the reservoir to a final concentration of 100 μM of each dNTP. Temperature maintained at 20° C. A voltage of 80 mV is applied and maintained and conductivity through the chip is measured over time as the polymerase processes the template nucleic acid according to its natural functions.

### Example 6

### ASSEMBLY AND USE OF A LOW-NOISE SOLID-STATE NANOSENSOR COMPLEX COMPOSED OF A DNA EXONUCLEASE POLYNUCLEOTIDE BINDING PROTEIN AND A SOLID-STATE CHIP

This Example describes how a DNA exonuclease PBP may be assimilated with a low-noise solid-state support chip to form a nanosensor complex for DNA sequencing applications. In this Example, PBP is the phage lambda DNA exonuclease with inherent 5′ to 3′ exonuclease activities. First, a low capacitive solid-state chip is fabricated starting from a silicon chip with dimensions of 200 μm×10 μm. The chip is cleaned using the RCA process and then the following coatings are applied to the chip: 1) 30 nm LPCVP silicon (Si) lean silicon nitride (SiN) on both sides; 2) 3 μm PECVD SiO₂ on the backside of the chip; 3) 200 nm PECVD SiN on the backside of the chip. Lithography masking technology is then used to RIE etch wells of 30 nm into the SiN on the frontside of the chip. Lithography masking technology is then further used to RIE etch wells of 200 nm on the on the backside of the chip. Finally, KOH aniso/isotropic etching is used to create the geometry of the chip depicted in FIG. 6, which illustrates SiN mask 610, Si substrate 620, SiN membrane 630, SiO2 layer 640, and SiN mask 650. The support pore 660, 4 nm in diameter, is drilled into the 30 nm thick silicon nitride membrane as denoted by the arrow using a FEI Technai-transmission electron microscope. FIG. 7 depicts an electron micrographic image of an electroconductive pore drilled into a solid-state silicon chip according to the methods of the present invention. A test apparatus has 2 reservoirs filled with electrolyte solution which are separated by the silicon chip mounted on a gasket so that the only fluid connection between the reservoirs is through pore 660 located in the silicon nitride membrane of the chip. Each reservoir has a Ag/AgCl electrode through which potential is applied and current can be measured with a Molecular Devices Axopatch 200B amplifier.

A PBP-DNA template complex is generated next. In this Example, the phage lambda DNA exonuclease PBP and double-stranded DNA template are produced using standard molecular biology technologies. The 5′ ends of the double-stranded DNA template are phosphorylated using well known T4 Polynucleotide Kinase based methods. The resulting modified template is purified to using well known silica glass fiber methods. The double-stranded DNA template is incubated with the phage lambda DNA exonuclease in an aqueous solution containing 30 mM Tris-HCl, pH 7.5, 2 mM EDTA, 4 mM DTT, and 30 mM ammonium acetate, which binds the DNA template following its natural functions but does not initiate exonuclease digestion due to the lack of magnesium cofactor. The lambda exonuclease-DNA template assembly is then assimilated, or coupled, with the solid-state chip by adding the exonuclease assembly to the cis reservoir of the test apparatus solution containing an aqueous solution of 30 mM Tris-HCl, pH 7.5, 2 mM EDTA, 4 mM Dithiothreitol, and 300 mM Ammonium Acetate in the cis reservoir and an aqueous solution of 1000 mM NH4Cl on the trans side reservoir and applying a positive electric potential to the trans reservoir. The negatively charged DNA template molecule will then thread through the pore, thereby guiding and anchoring the DNA exonuclease complex into the chip.

While maintaining a positive trans side voltage bias, sequencing of the DNA template with the lambda DNA exonuclease solid-state nanosensor chip assembly is initiated by adding MgCl2, a cofactor necessary for exonuclease activity, to a final concentration of 10 mM in the cis reservoir. Temperature is maintained at 23° C. during the sequencing reaction. A voltage of 80 mV is applied and maintained and conductivity through the membrane is measured over time as the exonuclease processes the template nucleic acid according to its natural functions.

### Example 7

### ASSEMBLY AND USE OF A LOW-NOISE SOLID-STATE NANOSENSOR COMPLEX COMPOSED OF A DNA HELICASE POLYNUCLEOTIDE BINDING PROTEIN AND A SOLID-STATE CHIP

This Example describes how a DNA helicase PBP may be assimilated with a low-noise solid-state support chip to form a nanosensor complex for DNA sequencing applications. In this Example, the PBP is the DnaB-like helicase, bacteriophage T7 gp4, with inherent duplex strand separation activity. First, a low capacitive solid-state chip is fabricated starting from a silicon chip with dimensions of 200 μm×10 μm. The chip is cleaned using the RCA process and then the following coatings are applied to the chip: 1) 30 nm LPCVP silicon (Si) lean silicon nitride (SiN) on both sides; 2) 3 μm PECVD SiO₂ on the backside of the chip; 3) 200 nm PECVD SiN on the backside of the chip. Lithography masking technology is then used to RIE etch wells of 30 nm into the SiN on the frontside of the chip. Lithography masking technology is then further used to ME etch wells of 200 nm on the on the backside of the chip. Finally, KOH aniso/isotropic etching is used to create the geometry of the chip depicted in FIG. 6, which illustrates SiN mask 610, Si substrate 620, SiN membrane 630, SiO2 layer 640, and SiN mask 650. The support pore 660, 4 nm in diameter, is drilled into the 30 nm thick silicon nitride membrane as denoted by the arrow using a FEI Technai-transmission electron microscope. FIG. 7 depicts an electron micrographic image of an electroconductive pore drilled into a solid-state silicon chip according to the methods of the present invention.

A test apparatus has two reservoirs filled with electrolyte solution which are separated by the silicon chip mounted on a gasket so that the only fluid connection between the reservoirs is through pore 660 located in the silicon nitride membrane of the chip. Each reservoir has a Ag/AgCl electrode through which potential is applied and current can be measured with a Molecular Devices Axopatch 200B amplifier.

A PBP-DNA template complex is next generated. In this Example, the DNA helicase PBP and double-stranded DNA template are produced using standard molecular biology technologies. The double-stranded DNA template is incubated with the T7 gp4 DNA helicase in an aqueous solution containing 30 mM Tris-HCl, pH 7.5, 10 mM MgCl₂ 4 mM DTT, and 30 mM ammonium acetate, which binds the DNA template following its natural functions. The T7 gp4 helicase-DNA template assembly is then assimilated, or coupled, with the solid-state chip by adding the assembly to the cis reservoir of the test apparatus containing an aqueous solution of 30 mM Tris-HCl, pH 7.5, 10 mM MgCl2, 4 mM Dithiothreitol, and 300 mM Ammonium Acetate in the cis reservoir and an aqueous solution of 1000 mM NH4Cl on the trans side reservoir. Applying a positive electric potential across the membrane threads the negatively charged DNA template molecule through the pore, thereby guiding and anchoring the DNA exonuclease into the solid-state pore.

While maintaining a positive trans side voltage bias, sequencing of the DNA template with the T7 gp4 DNA helicase solid-state nanosensor chip assembly is initiated by adding ATP to the cis reservoir. Temperature is maintained at 23° C. during the sequencing reaction. A voltage of 80 mV is applied and maintained and conductivity through the membrane is measured over time as the helicase processes the template nucleic acid according to its natural functions.

### Example 8

### ASSEMBLY AND USE OF A NANOSENSOR COMPLEX COMPOSED OF A DNA POLYMERASE POLYNUCLEOTIDE BINDING PROTEIN EMBEDDED IN A LIPID BILAYER MEMBRANE

This Example describes how a DNA polymerase PBP may be assimilated with a lipid bilayer membrane by embedding the PBP directly in the membrane to form a nanosensor complex for DNA sequencing applications. In this Example, the PBP is the Phi29 DNA polymerase with inherent polynucleotide strand-displacement and exonuclease activities. First, a lipid bilayer membrane is formed with the lipid 1,2-diphytanoyl-sn-glycero-3-phosphoethanolamine (C₄₅H₉₀NO₈P). Briefly, as described previously, the lipid bilayer is formed over an aperture in a PTFE solid support cell by priming the cell with a thin coat of lipid dissolved in hexane and coating over the support cell. Hexane is removed by air-drying the painted cell and a lipid membrane is painted over the support cell by dissolving PE in 1-hexadecene and depositing the solution over the primed support cell with a pipette and moving an air bubble over the aperture in the support cell to form a lipid bilayer membrane over the aperture.

As DNA polymerases are not normally transmembrane proteins, the Phi29 DNA polymerase is genetically engineered to introduce hydrophobic amino acids (i.e. “groups”) on at least one exterior surface of the protein. The crystal structures of many DNA polymerases, including the Phi29 polymerase, have been solved and it is straightforward in the art to predict which amino acids may be targeted for mutation to create hydrophobic patches to facilitate membrane insertion. An aqueous solution of the genetically modified Phi29 DNA polymerase is added to the lipid bilayer and the modified PBP is allowed to self-assemble on the membrane and insert to form a transmembrane pore. Insertion is promoted using a well known method of electroporation by which voltage pulses are applied across the bilayer to create transient openings for insertion.

The transmembrane Phi29 DNA polymerase PBP, following its natural functions, is then complexed with double-stranded DNA template to which an oligonucleotide primer is duplexed by adding the primed template to the cis reservoir that the transmembrane polymerase presents to and an aqueous solution containing 30 mM Tris-HCl, pH 7.5, 10 mM MgCl₂, 4 mM DTT, and 30 mM ammonium acetate. An electric potential is applied across the membrane to thread the negatively charged DNA template molecule through the pore provided by the DNA polymerase, thereby guiding and anchoring the DNA template in the polymerase nanosensor.

While maintaining a positive trans side voltage bias, sequencing of the DNA template with the Phi29 DNA polymerase nanosensor is initiating by adding a mixture of all 4 deoxynucleotide triphosphate substrates to the cis side reservoir to a final concentration of 100 uM of each dNTP. Temperature of the sensor is maintained at 23° C. during the sequencing reaction. A voltage of 80 mV is applied and maintained across the membrane while conductivity through the membrane is measured over time as the polymerase processes the template nucleic acid according to its natural functions.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, including U.S. Provisional Application No. 62/127,464, filed on Mar. 3, 2015, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A system for determining the nucleotide sequence of a polynucleotide in a sample, the system comprising:
a cis chamber, a trans chamber, wherein the cis and trans chambers are separated by a membrane and wherein the cis and trans chambers include an electrically conductive mixture;
a polynucleotide binding protein assimilated with the membrane to form an electroconductive pore therein, wherein the polynucleotide binding protein provides a constriction site in the pore, and wherein the constriction site undergoes conformational changes in response to processing of a membrane spanning target polynucleotide by the polynucleotide binding protein;
drive electrodes in contact with the electrically conductive reaction mixture on either side of the membrane for producing a voltage drop across the pore;
one or more measurement electrodes connected to electronic measurement equipment for measuring ion current through the pore; and
a computer to translate the ion current measurement into nucleic acid sequence information.

2. The system of claim 1, wherein the reaction mixture comprises reagents necessary for polynucleotide binding and processing.

3. The system of claim 1, wherein the polynucleotide binding protein is selected from the group consisting of helicases, DNA polymerases, RNA polymerases, exonucleases, endonucleases, and transcription factors.

4. The system of claim 3, wherein the polynucleotide binding protein is a helicase.

5. The system of claim 4, wherein the helicase is a DnaB-like helicase.

6. The system of claim 3, wherein the polynucleotide binding protein is a DNA polymerase.

7. The system of claim 3, wherein the polynucleotide binding protein is an exonuclease.

8. The system of claim 1, wherein the membrane is comprised of amphiphilic molecules.

9. The system of claim 8, wherein the amphiphilic molecules form a lipid bilayer.

10. The system of claim 1, wherein the membrane is a solid-state membrane.

11. The system of claim 10, wherein the polynucleotide binding protein is assimilated with a support pore preformed in the membrane.

12. The system of claim 9, wherein the polynucleotide binding protein is assimilated with a support pore embedded in the bilayer.

13. The system of claim 12, wherein the support pore is a natural pore forming protein.

14. The system of claim 9, wherein the polynucleotide binding protein is assimilated with the lipid bilayer by embedding the protein in the bilayer.

15. The system of claim 14, wherein the polynucleotide binding protein is genetically modified to introduce hydrophobic groups on at least one outer surface of the protein.

16. A method for determining the nucleotide sequence of a polynucleotide in a sample, the method comprising the steps of:
providing a membrane having at least one polynucleotide binding protein assimilated therein to form an electroconductive pore, wherein the polynucleotide binding protein provides a constriction site in the pore, and wherein the constriction site undergoes conformational changes in response to processing of a membrane spanning target polynucleotide by the polynucleotide binding protein;
contacting the polynucleotide binding protein with an electrically conductive reaction mixture comprising reagents required for polynucleotide processing by the polynucleotide binding protein;
providing a voltage drop across the membrane that induces ion current through the constriction site that is modulated by polynucleotide binding protein processing of the membrane spanning polynucleotide;
measuring the resulting base-specific ion current over time, thus determining sequence information about the polynucleotide.

17. The method of claim 16, wherein the resulting base-specific ion current comprises the magnitude of the ion current through the constriction site.

18. The method of claim 16, wherein the resulting base-specific ion current comprises the shape of the measured ion current through the constriction site over time. 19 The method of claim 16, wherein the polynucleotide binding protein is selected from the group consisting of helicases, DNA polymerases, RNA polymerases, exonucleases, endonucleases, and transcription factors.

20. The method of claim 19, wherein the polynucleotide binding protein is a helicase.

21. The method of claim 20, wherein the helicase is a DnaB-like helicase.

22. The method of claim 19, wherein the polynucleotide binding protein is an exonuclease.

23. The method of claim 20 or 22, wherein the polynucleotide comprises a double-stranded nucleic acid.

24. The method of claim 19, wherein the polynucleotide binding protein is a DNA polymerase.

25. The method of claim 24, wherein the polynucleotide comprises an oligonucleotide primer bound to a single stranded nucleic acid template.

26. The method of claim 16, wherein the membrane is comprised of amphiphilic molecules.

27. The method of claim 26, wherein the amphiphilic molecules form a lipid bilayer.

28. The method of claim 16, wherein the membrane is a solid-state membrane.

29. The method of claim 28, wherein the polynucleotide binding protein is assimilated with a support pore preformed in the membrane.

30. The method of claim 27, wherein the polynucleotide binding protein is assimilated with a support pore embedded in the bilayer.

31. The method of claim 30, wherein the support pore is a natural pore forming protein.

32. The method of claim 27, wherein the polynucleotide binding protein is assimilated with the bilayer by embedding the protein in the bilayer.

33. The method of claim 32, wherein the polynucleotide binding protein is genetically modified to introduce hydrophobic groups on at least one outer surface of the protein.

34. A method for determining the nucleotide sequence of a polynucleotide in a sample, the method comprising the steps of:
providing a solid-state membrane having at least one polynucleotide binding protein assimilated therein to form an electroconductive pore, wherein the polynucleotide binding protein provides a constriction site in the pore, and wherein the constriction site undergoes conformational changes in response to processing of a membrane spanning target polynucleotide by the polynucleotide binding protein;
contacting the polynucleotide binding protein with an electrically conductive reaction mixture comprising reagents required for polynucleotide binding protein processing of the membrane spanning polynucleotide by the polynucleotide binding protein;
providing a high frequency drive potential across the membrane;
measuring the resulting base-specific ion current over time, thus determining sequence information about the polynucleotide.

35. A method for determining the nucleotide sequence of a polynucleotide in a sample, the method comprising the steps of:
providing a membrane having at least one polynucleotide binding protein assimilated therein to form an electroconductive pore, wherein the polynucleotide binding protein provides a constriction site in the pore, and wherein the protein is complexed with a membrane spanning target polynucleotide;
contacting the polynucleotide binding protein with a reaction mixture comprising reagents required for polynucleotide processing by the polynucleotide binding protein;
providing an optically detectable agent to the reaction mixture on a first side of the membrane, wherein the agent is capable of flowing through the pore to the reaction mixture on a second side of the membrane;
measuring the concentration of the agent in the reaction mixture on the second side of the membrane over time to detect the nucleotide-dependent binding and processing using optical means; and
identifying the types of nucleotides bound and processed by the polynucleotide binding protein using concentration modulation characteristics, thus determining sequence information about the polynucleotide.

36. The method of claim 35, wherein the optical means measure the agent directly.

37. The method of claim 36, wherein the agent is fluorescein.

38. The method of claim 35, wherein the optical means measure the agent indirectly.

39. The method of claim 38, wherein the agent is calcium.

40. The method of claim 39, wherein the reaction mixture on the second side of the membrane further comprises a fluorescent calcium indicator probe.

41. The method of claim 40, wherein the fluorescent calcium indicator probe is selected from the group consisting of Fluo-3, Fluo-4, and Fluo-5.

42. The method of claim 35, wherein the membrane is comprised of amphiphilic molecules.

43. The method of claim 42, wherein the amphiphilic molecules form a lipid bilayer.

44. The method of claim 35, wherein the membrane is a solid-state membrane.

45. The method of claim 44, wherein the polynucleotide binding protein is assimilated with a pore preformed in the membrane.

46. The method of claim 43, wherein the polynucleotide binding protein is assimilated with a support pore embedded in the bilayer. 47 The method of claim 46, wherein the support pore is a natural pore forming protein.

48. The method of claim 43, wherein the polynucleotide binding protein is assimilated with the bilayer by embedding the protein in the bilayer.

49. The method of claim 48, wherein the polynucleotide binding protein is genetically modified to introduce hydrophobic groups on at least one outer surface of the protein.
